(19) United States
(12) Patent Application Publication
(10) Pub. No.: US 2007/0030448 A1
(43) Pub. Date: Feb. 8, 2007

(54) Illumination unit for fundus cameras and/or ophthalmoscopes

(76) Inventors: Detlef Biernat, Jena (DE); Uwe Mohrholz, Jena (DE); Frank Teige, Jena (DE)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(21) Appl. No.: 10/576,332

(22) PCT Filed: Oct. 16, 2004
(86) PCT No.: PCT/EP04/11684

§ 371 Date: Apr. 18, 2006

(30) Foreign Application Priority Data
Oct. 22, 2003 (DE) 103 49 091.4

(51) Int. Cl. A61B 3/14

(52) U.S. Cl. 351/206

(57) ABSTRACT

The present invention is directed to an optical device for the observation and documentation of the ocular fundus and is preferably provided for fundus cameras. In order to generate a uniform illumination of the fundus by transillumination of the sclera in the illumination unit, according to the invention, for fundus cameras and/or ophthalmoscopes, the light emitted by the illumination source is coupled into individual light-conducting fibers or bundles of light-conducting fibers which extend into the area of the front lens of the fundus camera and ophthalmoscope and whose fiber ends are formed in such a way that the exiting light is projected on and transilluminates the sclera. The discomfort caused to the patient by pupil-dilating means is avoided as are the risks associated with the placement of contact lenses. Another substantial advantage of the illumination unit according to the invention is the extremely uniform, large-area illumination of the fundus, so that a correspondingly large visual field of the fundus can be observed and also documented.

### CROSS-REFERENCE TO RELATED APPLICATIONS

This application claims priority of International Application No. PCT/EP2004/011684, filed Oct. 16, 2004 and German Application No. 103 49 091.4, filed Oct. 22, 2003, the complete disclosures of which are hereby incorporated by reference.

### BACKGROUND OF THE INVENTION

a) Field of the Invention

The present invention is directed to an optical device with electronic or photographic image sensors for observing and documenting the ocular fundus. The device is preferably provided for fundus cameras and/or ophthalmoscopes. The diagnosis of diseases of the ocular fundus can be considerably facilitated in this way both for the examiner and for the patient.

b) Description of the Related Art

In most of the fundus cameras known from the art, illumination, observation and/or documentation is carried out through the pupil of the eye of the patent being examined. For this purpose, the pupil is always dilated to a certain extent.

Normally, when a fundus camera is used, the pupil of the patient must be dilated medicinally. It is known from the prior art that when the ocular fundus is illuminated by infrared (invisible) light, there is no pupillary reflex on the part of the patient and, in a darkened room, dilation of the pupil occurs without the use of medication. This principle is put to use in the non-mydriatic fundus camera. When the pupil is sufficiently dilated, the eye is briefly illuminated by white (visible) light in order to record an image of the ocular fundus. In a non-mydriatic fundus camera, because of its operating principle, infrared light is used for observation and the final image is recorded in white light of short wavelength.

In U.S. Pat. No. 4,200,362, O. Pomerantzeff describes an ophthalmoscope with a large illumination field. In a first embodiment, there are two separate annular arrangements of light-conducting fibers around the contact lens that is to be arranged on the patient's cornea in order to achieve the largest possible and most uniform possible illumination of the fundus. Since the illumination, observation and documentation of the fundus must be carried out through the pupil of the eye being examined, the pupil must be extremely dilated. Apart from the fact that the arrangement requires very precise construction, placement of the contact lens on the cornea of the eye to be examined presents additional difficulties. The contact lens must be placed on the dilated pupil very accurately in order to prevent glare.

In a second embodiment, the illumination of the fundus is carried out through the sclera of the eye. The illumination light is scattered by the sclera and accordingly illuminates the entire fundus. The pupil needs to be dilated only slightly or not at all, since now only observation and documentation are performed through the pupil.

However, these solutions are disadvantageous in that illumination, observation and documentation are carried out by contact methods, i.e., by fitting contact lenses and other optical devices. Correspondingly strict requirements are imposed with respect to the cleanliness (sterility) of the contact surfaces.

An apparatus for examining the ocular fundus by which a large area of the fundus can be observed and documented without a scanning movement is proposed by E. Svetliza in U.S. Pat. No. 5,966,196. Here again light-conducting fibers are provided in an annular arrangement around the observation optics to generate a uniform illumination of the fundus. The illumination distribution can be adapted individually by controlling the illumination source.

While observation and documentation take place without direct contact with the cornea, illumination in the contact method is carried out by placing the ends of the fibers on the cornea of the eye to be examined in order to allow individual adaptation to the curvature of the eye, for example, in small children. While use of pupil-dilating agents can be dispensed with in the described solution, placement of the ends of the fibers of the illumination unit and the correspondingly demanding requirements with respect to cleanliness (sterility) of the contact surfaces are disadvantageous.

### OBJECT AND SUMMARY OF THE INVENTION

It is the primary object of the present invention to improve the observation and documentation of the ocular fundus by means of an ophthalmologic device, particularly a fundus camera, in such a way that the discomfort of and risk to the patient can be minimized.

According to the invention, this object is met by an illumination unit for fundus cameras and/or opthalmoscopes having a front lens for generating a uniform illumination of the fundus by transillumination of the sclera. The illumination unit comprises an illumination source for emitting light, where the light emitted by the illumination source is coupled into individual light-conducting fibers or bundles of light-conducting fibers which extend into the area of the front lens of the fundus camera and ophthalmoscope. Ends of the fibers are formed in such a way that the exiting light is projected on the sclera of the eye to be examined and transilluminates the sclera.

In a fundus camera according to the invention, observation is carried out, as usual, through the patient's pupil, but without having to dilate the pupil. The required optical arrangement for observing and documenting the fundus corresponds to that of a conventional fundus camera. However, the illumination principle used for this purpose deviates from that of a conventional fundus camera. In contrast to non-mydriatic fundus cameras, white light can be used for illumination. Further, the illumination light is not projected into the patient's eye through the patient's pupil but through the sclera.

The light of the light source for observation and documentation is preferably coupled into bundled light-conducting fibers by suitable optical systems. The light exits in the area of the front lens of the fundus camera via two or more end pieces of the light-conducting fiber bundle. Suitable optical projection systems in front of the end pieces of the light-conducting fiber bundle project the exiting light onto the sclera of the patient's eye. An optimal illumination of the fundus is achieved by coupling in the illumination light in the area of the pars plana, that is, between the cilliary body and the ora serrata, because the transmission of the sclera for the illumination light being used is relatively high at that area.

The arrangement according to the invention preferably uses two fiber end pieces which couple the illumination light into the nasal and temporal area of the above-mentioned portion of the sclera.

The diagnosis of diseases of the ocular fundus can be considerably facilitated for both the examiner and the patient by a fundus camera based on the fundamental inventive idea.

The invention will be described more fully in the following with reference to an embodiment example shown in the drawing.

### BRIEF DESCRIPTION OF THE DRAWINGS

In the drawings:

FIG. 1 is a top view illustrating the basic construction of a fundus camera with the illumination unit according to the invention; and

FIG. 2 shows a variant of the above-mentioned fundus camera with additional pulsed light source for documentation.

### DESCRIPTION OF THE PREFERRED EMBODIMENTS

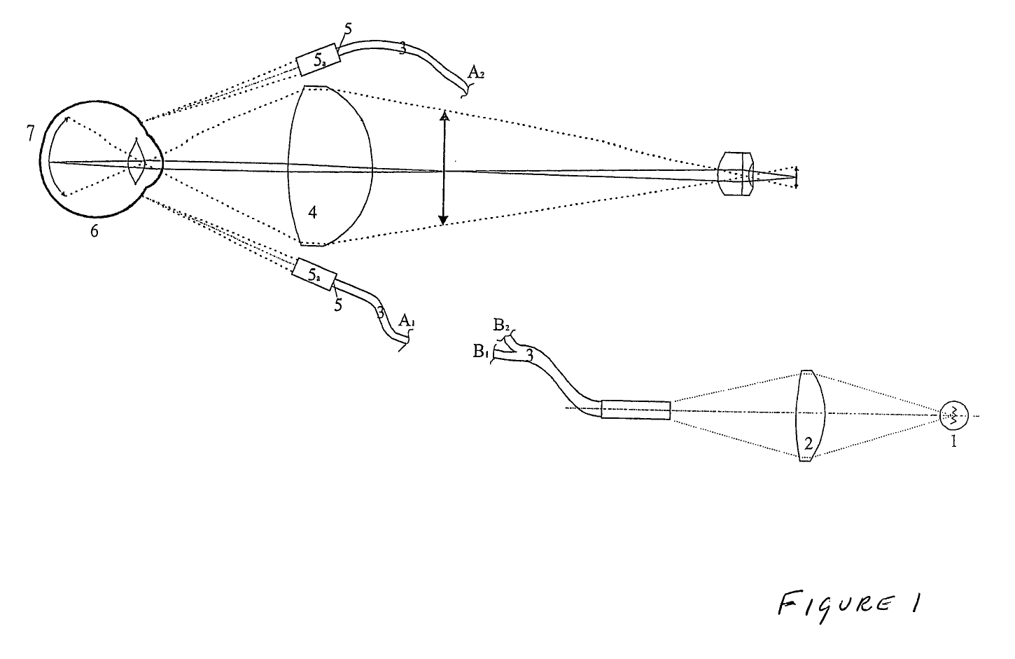

The illumination unit, according to the invention, for fundus cameras and/or ophthalmoscopes serves to generate a uniform illumination of the fundus by transillumination of the sclera. FIG. 1 shows a top view of the basic construction of a fundus camera with the illumination unit according to the invention. The light emitted by the illumination source 1 is coupled by optical means 2 into light-conducting fibers 3 which extend to the area of the front lens 4 of the fundus camera or ophthalmoscope and whose fiber ends 5 are formed in such a way that the exit faces are projected by means of an optical system 5a onto the sclera of the eye 6 being examined and transilluminate the sclera. However, it is also possible, in principle, to use bundles of light-conducting fibers 3.

At least two individual light-conducting fibers 3 or bundles of light-conducting fibers 3 are preferably provided and arranged in such a way that the light emitted by the illumination source 1 penetrates the sclera in the nasal and temporal area. The optimal illumination of the fundus 7 is achieved when the light of the illumination source 1 is coupled in in the area of the pars plana, that is, in a substantially annular surface between the cilliary body and the ora serrata (retina). The optical transmission of the sclera for the light of the illumination source 1 employed is greatest in the area of the pars plana and reaches approximately 50%. In contrast to non-mydriatic fundus cameras, white light is used.

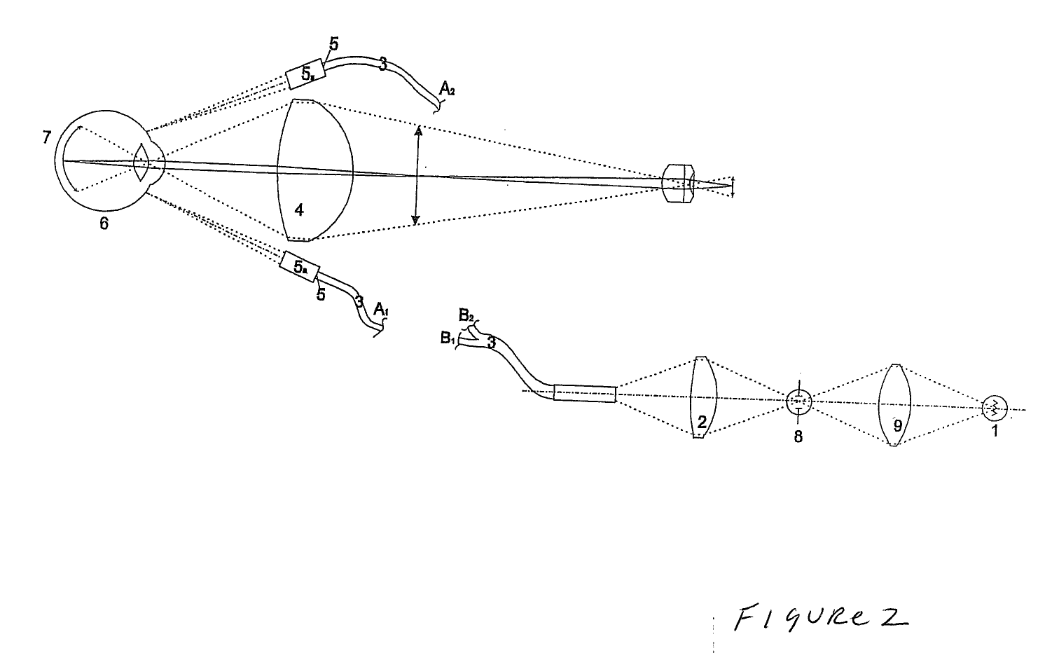

For fundus cameras and/or ophthalmoscopes which are used only for observation of the fundus 7, an illumination source 1 for continuous illumination of the sclera is sufficient. A suitable continuously radiating halogen lamp can preferably be used for this purpose. For fundus cameras and/or ophthalmoscopes which additionally have devices for documentation, an additional pulsed light source 8 is provided for electronic and/or photographic documentation in addition to the illumination source 1 for continuous illumination of the sclera. FIG. 2 shows a basic construction of this kind. The light of the continuous illumination source 1 is imaged in the focal plane of the pulsed light source 8 by optical means 9.

In a particularly advantageous manner, the inventive illumination unit affords the possibility that the ends 5 of the light guides located in the area of the front lens 4 can be moved separately or jointly with the optical system 5a for projection independent from the position of the fundus camera. In this way, for example, when the observation unit is exactly oriented, the illumination distribution on the fundus can be changed, or any misadjustment of the illumination unit can be corrected.

With the arrangement according to the invention, the diagnosis of diseases of the ocular fundus is substantially facilitated for the examiner and for the patient.

Since the illumination of the fundus is carried out through the sclera, dilation of the patient's pupil by medicinal means is superfluous. The pupil diameter of about 2 mm that is required for observation is present in all patients even in normal ambient illumination.

The discomfort caused to the patient by pupil-dilating means is avoided as are the risks involved in the placement of contact lenses:

Another substantial advantage of the illumination unit according to the invention is the extremely uniform, large-area illumination of the fundus, so that a correspondingly large visual field of the fundus can be observed and also documented.

In contrast to illumination through the dilated pupil of an eye being examined, the requirements for accurate positioning of the ends of the light-conducting fibers are not as demanding because the illumination light is scattered by the sclera and is accordingly distributed over the entire fundus.

While the foregoing description and drawings represent the present invention, it will be obvious to those skilled in the art that various changes may be made therein without departing from the true spirit and scope of the present invention.

What is claimed is:

1-4. (canceled)

5. An Illumination unit for fundus cameras and/or ophthalmoscopes having a front lens for generating a uniform illumination of the fundus by transillumination of the sclera, comprising:
an illumination source for emitting light;
the light emitted by the illumination source being coupled into individual light-conducting fibers or bundles of light-conducting fibers which extend into the area of the front lens of the fundus camera and ophthalmoscope; and
ends of said fibers being formed in such a way that the exiting light is projected on the sclera of the eye to be examined and transilluminates the sclera.

6. The illumination unit according to claim 5, wherein at least one light-conducting fiber or bundle of light-conducting fibers is provided and arranged in such a way that the light of the illumination source transilluminates the sclera in the nasal and temporal area.

7. The illumination unit according to claim 5, wherein an additional pulsed light source is provided for electronic and/or photographic documentation in addition to the illumination source for continuous illumination of the sclera for purposes of observation, wherein the light of the continuous illumination source is imaged in the focal plane of the pulsed light source by optical means.

8. The illumination unit according to claim 5, wherein the ends of the light-conducting fibers or light-conducting fiber bundle located in the area of the front lens can be moved separately or jointly independent from the position of the fundus camera.
